(19) United States
(12) Patent Application Publication
(10) Pub. No.: US 2015/0316544 A1
(43) Pub. Date: Nov. 5, 2015

(54) RELEASABLE MAGNETIC CELL CAPTURE SYSTEM

(71) Applicant: NORTHEASTERN UNIVERSITY, Boston, MA (US)

(72) Inventors: Mansoor M. Amiji, Attleboro, MA (US); Amit SINGH, Melrose, MA (US)

(21) Appl. No.: 14/653,410

(22) PCT Filed: Jan. 14, 2014
(86) PCT No.: PCT/US2014/011497

§ 371 Date: Jun. 18, 2015

Related U.S. Application Data
(60) Provisional application No. 61/752,281, filed on Jan. 14, 2013.

(51) Int. Cl. G01N 33/543; G01N 33/574

(52) U.S. Cl. CPC G01N 33/5434; G01N 33/57492; G01N 2333/71; G01N 2446/20

(57) ABSTRACT

Compositions and methods for capturing specific cell types from a mixture in a liquid suspension of cells are provided. Targeted magnetic nanoparticles of the invention can be utilized to isolate, quantify, and characterize circulating tumor cells from complex body fluids, such as blood. The magnetic nanoparticles are releasable after targeted cells have been isolated, leaving the cells in a viable state for characterization and growth in culture. The targeted magnetic nanoparticles are fabricated using surface-functionalized super paramagnetic nanoparticles that are encapsulated in a biodegradable and biocompatible polymer to form microparticles. The microparticles are rendered target-selective by additional coatings of gelatin and gold nanoparticles which are derivatized with a targeting ligand specific for a targeted cell type.

### CROSS-REFERENCE TO RELATED APPLICATIONS

This application claims the priority of U.S. Provisional Application No. 61/752,281, filed Jan. 14, 2013, entitled “RELEASABLE MAGNETIC CELL CAPTURE SYSTEM”, which is hereby incorporated herein by reference in its entirety.

### BACKGROUND

Metastasis is a hallmark of cancer, depicting the invasive behavior of the disease and is the prime cause of intractable morbidity and death in 90% of cancer patients. It is a complex physiological phenomenon involving detachment of cells from tumor, their epithelial to mesenchymal transition (EMT), intravasation into the vascular system, embolization, extravasation to distant organs, mesenchymal to epithelial transition (MET), and formation of micrometastases at distant sites resulting in tumorous growth (Paterlini-Brechotand and Benali, 2007). See FIG. 1. The invasiveness of malignant tumors is effected by circulation of a proportion of tumor cells (CTCs) or microemboli (CTMs) and their presence in peripheral blood has been confirmed in all major carcinomas (Allard et al., 2004). It has further been ascertained that the number of CTCs in the peripheral blood circulation directly correlates to the metastatic progression of the tumor (Cohen et al., 2008) and is now considered as a surrogate prognostic cancer biomarker (Danila et al., 2011). Accurate counting of CTCs in blood has gained clinical relevance as a prognostic marker for assessment of tumor sensitivity to a therapeutic regimen, diagnosis of residual disease, and for devising a personalized therapy. Further, contrary to the belief that CTC dissemination is a late process in the progression of malignancy, studies have indicated that CTCs can be detected in peripheral circulation even during early stages of the disease (Nakagawa et al., 2007; Husemann et al., 2008).

Previous technologies for the capture and subsequent enumeration of CTCs have largely relied on physical properties, such as size, density, or charge, or molecular expression profile. Since more than 90% of tumors are epithelial in origin, epithelial cell adhesion molecule (EpCAM) is a commonly used biomarker for positive selection of CTCs in blood. CTC enrichment by negative selection can be achieved by removing CD45 positive cells from blood. A system that uses anti-EpCAM antibody for CTC capture from a patient's blood, diamidino-2-phenylindole (DAPI) stain to identify viable cells, and phycoerythrin labeled anti-cytokeratin antibody for cell counting is known as CellSearch®. The CellSearch® system has been approved by the US Food and Drug Administration as a stand-alone platform for monitoring progression-free survival and overall survival in patients with metastatic breast, colon, and prostate cancer, and this technology has been successfully validated (N. Lopez-Riquelme et al., 2013; E. Le Rhun et al., 2013; Peeters et al., 2013; Bozec et al., 2013; Muller et al., 2013; Ali et al., 2011; Andreopoulou et al., 2012; Naoe et al., 2012). Isolation by size of epithelial tumor (ISET) is a simple and straightforward approach for CTC enrichment or capture based on cell size, where 6-10 μm filters are used (Ferace et al., 2011). Density gradient centrifugation is yet another method that partitions CTCs from other blood cells based on the difference in their densities, and has led to development of technologies such as Oncoquick® (Muller et al., 2005) and Ficoll-Hypaque (Fizazi et al., 2007). Recent advancement in micro/nano fabrication methodologies combined with improved understanding of fluid dynamics has led to reports on capturing and enumeration of CTCs in microfluidic devices. Micropost CTC-Chip (μapCTC-Chip) relies on a laminar flow of blood through anti-EpCAM antibody-coated microposts for CTC capture (Nagrath et al., 2007), while the herringbone CTC-Chip (HbCTC-Chip) employs herringbone-shaped groves to direct cells towards antibody-coated surfaces (Stott et al., 2010). A microfluidic device with capability of both positive and negative selection for efficient isolation of CTCs has also been developed (Ozkumar et al., 2013).

Existing technologies that rely on anti-EpCAM antibody to target epithelial cells may not be optimal in view of numerous reports regarding differential expression levels of EpCAM in cancer cells (Litvinov et al., 1996; Braun et al., 1999; Thurm et al., 2003). Alternative methods based on physical properties and molecular analysis, on the other hand, are cumbersome, somewhat non-specific, and time-consuming Importantly, although existing technologies can harvest CTCs from the blood, the captured CTCs cannot be released from the capture surface for any further analysis. The ability to capture CTCs and subsequently release them without changing their cellular characteristics would offer significant advantage, since it would make it possible to perform genetic and molecular level analysis to understand metastatic processes, and would be useful in the design of novel therapeutic approaches to specifically target these processes in individual patients.

The ability to capture CTCs from blood is largely hampered by the extremely low level of their occurrence in the circulation, especially during early stages of metastatic progression, where they typically are present at a density of about 1-100 CTCs for every 10⁹ blood cells. Tremendous morphological heterogeneity and differences in the level of expression of molecular signature have been observed in CTCs originating from different organ sources as well as among CTCs originating from the same tumor type in different patients (Sidransky, 2002). These subtle differences in CTCs further compound the challenges associated with efficient capture.

Therefore, there remains a need for a general approach to harvesting CTCs that uses the full potential of cancer cell heterogeneity in surface biomarker expression, and the nuances of particle surface science, to develop a releasable CTC capturing system.

### SUMMARY OF THE INVENTION

Compositions and methods for capturing specific cell types from a mixture in a liquid suspension of cells are provided. Targeted magnetic nanoparticles of the invention can be utilized to isolate, quantify, and characterize circulating tumor cells from complex body fluids, such as blood. The magnetic nanoparticles are releasable after targeted cells have been isolated, leaving the cells in a viable state for characterization and growth in culture. The targeted magnetic nanoparticles are fabricated using surface-functionalized super paramagnetic nanoparticles that are encapsulated in a biodegradable and biocompatible polymer to form microparticles. The microparticles are rendered target-selective by additional coatings of gelatin and gold nanoparticles which are derivatized with a targeting ligand specific for a targeted cell type.

One aspect of the invention is a releasable targeted magnetic microparticle for use in capturing cells of a targeted type. The microparticle includes: a plurality of hydrophobic superparamagnetic nanoparticles embedded in a hydrophobic polymer to form a hydrophobic core microparticle; a coating of biopolymer surrounding the core microparticle; and a plurality of targeting nanoparticles forming a shell surrounding the core microparticle, wherein the targeting nanoparticles comprise a releasable targeting moiety on their surface.

In embodiments of the magnetic microparticle, the superparamagnetic nanoparticles include a material selected from iron, cobalt, nickel, salts or oxides thereof, and combinations thereof. In embodiments the hydrophobic polymer is PCL. In embodiments the biopolymer is thiolated gelatin. In embodiments the releasable targeting moiety is selected from the group consisting of: amino acids, peptides, proteins, sugars, oligosaccharides, nucleic acids, nucleotides, oligonucleotides, and aptamers. In embodiments the releasable targeting moiety is EGF or a derivative of EGF that binds to an EGF receptor. In embodiments the targeting moiety is coupled to the targeting nanoparticles by a thiol linkage. In embodiments the microparticle is releasable from its target cell by a disulphide bond disruptive agent, such as glutathione. In embodiments the microparticle is targeted to a circulating tumor cell. In embodiments the targeting nanoparticles comprise or consist of gold.

Another aspect of the invention is a composition containing a plurality of the magnetic microparticles described above. In embodiments the composition further includes a physiological buffer solution in which the magnetic microparticles are suspended. In embodiments the average particle diameter of the microparticles is in the range from about 500 nm to about 2000 nm In embodiments the average particle diameter of the microparticles is in the range from about 500 nm to about 1000 nm. In embodiments the average particle diameter of the microparticles is in the range from about 600 nm to about 800 nm.

Yet another aspect of the invention is a composition containing the releasable magnetic microparticle described above which is bound to a cell targeted by the microparticles of the composition.

Still another aspect of the invention is a method of isolating a cell. The method includes the steps of: providing a composition containing a plurality of the microparticles described above; contacting the composition with a fluid containing individual cells in suspension, whereby targeted cells bind to said magnetic microparticles; and isolating the magnetic microparticles and bound cells using a magnet. In embodiments of the method the targeted cells are circulating tumor cells and the fluid is a bodily fluid from a mammalian subject. In embodiments of the method the subject is a human cancer patient. In embodiments of the method the superparamagnetic nanoparticles contain iron oxide, the hydrophobic polymer comprises or consists of PCL, the biopolymer coating comprises or consists of gelatin or thiolated gelatin, and the microparticle is releasable from its target cell by a disulphide bond disruptive agent.

Another aspect of the invention is a method of fabricating a plurality of the releasable magnetic nanoparticles described above. The method includes the steps of: (a) providing a plurality of hydrophobic superparamagnetic nanoparticles; (b) embedding the nanoparticles of (a) in a matrix of hydrophobic polymer to form microparticles containing the nanoparticles; (c) coating the microparticles from (b) with a biopolymer to form a shell of the biopolymer surrounding the microparticles; and (d) attaching a plurality of targeting nanoparticles to the biopolymer shell of (c) to form the plurality of the releasable magnetic nanoparticle, wherein the targeting nanoparticles contain a plurality of targeting moieties attached to their surface. In embodiments of the method, the method further includes washing and/or isolating the microparticles of any of steps (b)-(d) using a magnet. In embodiments of the method, the method further includes suspending the microparticles resulting from step (d) in a physiological buffer solution.

Yet another aspect of the invention is a microparticle containing a plurality of hydrophobic superparamagnetic nanoparticles embedded in a hydrophobic polymer. In embodiments of the miceoparticle, the nanoparticles are uniformly distributed within the microparticle. In embodiments the nanoparticles comprise or consist of a material selected from iron, cobalt, nickel, salts or oxides thereof, and combinations thereof. In preferred embodiments, the material is iron oxide. In embodiments of the microparticle the hydrophobic polymer is PCL. Another aspect of the invention is a composition containing a plurality of the microparticles described in the preceding paragraph.

Still another aspect of the invention is a kit containing the targeted releasable microparticles described above and one or more reagents and/or instructions for use of the microparticles, such as their use in isolating a cell as described above.

Another aspect of the invention is a kit containing the microparticles described two paragraphs above and one or more reagents and/or instructions for use of the composition for fabricating the targeted releasable microparticles described above.

### BRIEF DESCRIPTION OF THE DRAWINGS

FIG. 1 is a schematic diagram of the events leading to the escape of primary tumor cells into the blood as CTCs and subsequent embolization and dissemination of the CTCs into secondary metastases.

FIG. 2 is a schematic diagram of a method of synthesizing target-specific particles (Au@PCL-SPION) for reversible magnetic harvesting of selected cell types. Small paramagnetic iron oxide nanoparticles (SPION) are encapsulated with poly(ε-caprolactone) (PCL) to form PCL-SPION. The PCL-SPION particles are then coated with gold nanoparticles to which are attached a targeting moiety to form Au@PCL-SPION, which are used to bind and capture tumor cells. The tumor cells can be subsequently released by adding a competitive binding agent. The released cells maintain their phenotypic profile and viability.

FIG. 3A shows a transmission electron micrograph (TEM) of oleic acid-capped SPION particles. FIG. 3B shows a TEM of poly(e-caprolactone) (PCL) microparticles that encapsulate SPIONs in their core. The inset shows a high magnification image of a PCL-SPION. FIG. 3C shows a TEM of borohydride-reduced gold nanoparticles. FIG. 3D shows a TEM of gold nanoparticle-capped PCL microparticles.

FIG. 4A shows a TEM of gelatin-coated PCL-SPIONs that have been further coated with gold nanoparticles (Au@ Gelatin@PCL-SPION). FIG. 4B shows a scanning tunneling electron micrograph (SEM) of Au@ Gelatin@PCL-SPION particles obtained using secondary electrons. FIG. 4C shows an SEM image of Au@ Gelatin@PCL-SPION particles obtained using backscatter electrons.

FIG. 5A shows a bright field microscope image (20× magnification) of control Panc-1 (luc) cells. FIG. 5B shows a bright field microscope image (20× magnification) of Pane-1 (luc) cells captured, purified, and released from a blood sample using targeted

Au@ Gelatin@PCL-SPION magnetic nanoparticles (MNPs) functionalized with peptide, or t-MNPs.

FIGS. 6A, 6B, and 6C present bright field microscopic images of luciferase-expressing Panc-1 human pancreatic adenocarcinoma cells captured and released from blood spiked with 5000, 500, and 50 cells per ml blood, respectively. FIGS. 6D, 6E, and 6F show bioluminescence images of luciferase-expressing Panc-1 human pancreatic adenocarcinoma cells captured and released from blood spiked with 5000, 500, and 50 cells per ml blood, respectively. Bioluminescence imaging was performed on cells after they were sub-cultured for 10 days to show that they remained viable and continued to express the stably transfected reporter enzyme.

FIG. 7 is a histogram plot demonstrating the cell harvesting efficiency of polymeric micro-magnets. Different numbers of luciferase-expressing Panc-1 cells were spiked in 1 mL of whole blood and the number of cells harvested was quantified.

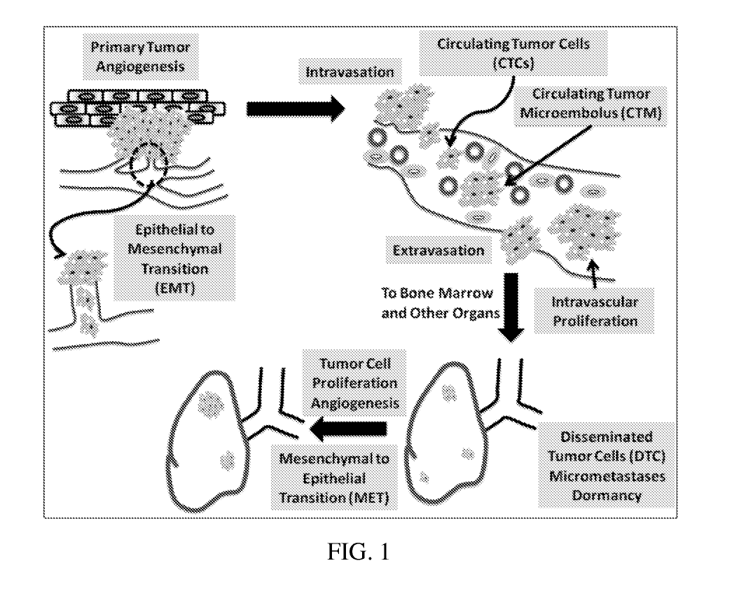

### DETAILED DESCRIPTION OF THE INVENTION

The invention provides compositions and methods for capturing specific cell types from a mixture in a liquid suspension of cells. The compositions and methods of the invention can be utilized, for example, to isolate, quantify, and characterize circulating tumor cells (CTCs) from complex body fluids. The invention achieves this through the releasable target-selective magnetic nanoparticles described herein. The approach is based on using surface-functionalized super paramagnetic iron oxide nanoparticles (SPION) that are encapsulated in a biodegradable and biocompatible polymer to form microparticles (micro-magnets). The micromagnets are rendered target-selective by additional coatings of gelatin and gold nanoparticles which are derivatized with a targeting ligand specific for a targeted cell type.

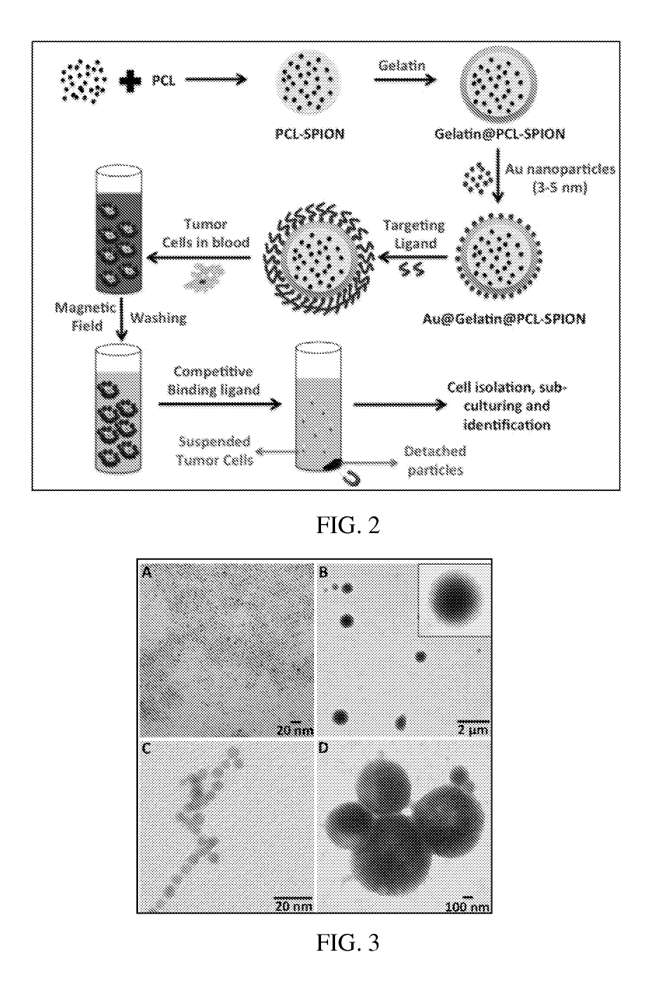

The general approach for preparing releasable targeted micromagnets of the invention is shown in FIG. 2. First, SPIONs are encapsulated in oleic acid to form small microparticles having a hydrophobic outer shell and evenly distributed (i.e., not aggregated) super paramagnetic iron oxide (Fe₂O₃) nanoparticles (SPIONs) within.

The oleic acid-capped hydrophobic SPIONs are then encapsulated in poly(ε-caprolactone) (PCL) particles, thereby generating PCL-SPION microparticles. PCL is an FDA-approved biodegradable polyester that has been used in various biomedical applications for drug delivery (Shenoyand et al., 2005; Chawlaand et al., 2003; and Chawlaand et al, 2003), and in tissue engineering (Rezwan et al., 2006). PCL is hydrophobic, and the hydrophobic core of PCL microparticles serves as an ideal reservoir for hydrophobic materials. Therefore, PCL particles can be loaded with oleic acid-capped SPIONs to form magnetic field responsive PCL microparticles.

Next, a coating of thiolated gelatin is applied onto the SPION-encapsulated PCL particles. Thiolated gelatin facilitates binding of gold nanoparticles on the surface of gelatin coated SPIONs. Accordingly, the gelatin-coated SPIONs can be coated with covalently attached gold nanoparticles.

The surface of the gold nanoparticles can be used to functionalize the particles by attaching a targeting moiety, such as a peptide to the gold surface using, for example, a thiol linkage. The targeting moiety provides for specific binding of the particles to cells expressing receptors specific to the targeting moiety. For example, a peptide that specifically binds to epidermal growth factor receptor (EGFR), which is over-expressed on the surface of several tumor cells, can be used to target the particles to EGFR-overexpressing tumor cells. Thiol-based binding of peptide is reversible, and thus allows displacement of the bound peptide in the presence of a competitive ligand (Livand and Salmeron, 1994) such as a disulphide bond disruptive agent. An example of a competitive ligand for disrupting a peptide linkage to gold is glutathione, a naturally occurring tripeptide containing a thiol group. Glutathione is preferred for applications such as cell capture since it does not have cytotoxic effect on cells, and is present as an important component of cell growth media.

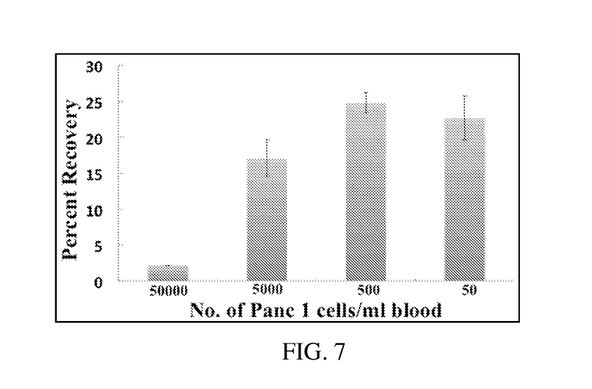

The gold coated surface of the PCL microparticles was functionalized with EGF receptor targeting peptide using gold-thiol chemistry, thereby generating targeted magnetic nanoparticles (t-MNPs) capable of specifically capturing cancer cells overexpressing EGFR. The size of the particles is controlled to be preferably in the range of 600-800 nm average diameter, since particles below 300 nm are known to be actively taken up by cancer cells. In model studies, cells captured by the t-MNPs could be successfully released by glutathione to displace the bound peptides on the particle surface (see results shown in FIG. 7).

Luciferase expressing Panc-1 cells were tested as a model cell system for demonstrating cell capture by the t-MNPs described herein. However, t-MNPs according to the invention can be used for any other EGFR-overexpressing cell, or cells expressing a sufficient density of selected target receptors on their surface. Results obtained established that the t-MNPs are capable of capturing cells present at a density as low as a few hundred cells/mL of blood.

It is noteworthy that the target cells captured by the t-MNPs could be released through competition with glutathione, which was used as a competitive ligand to disrupt the EGFR targeting peptide's linkage to the gold nanoparticles, thereby releasing the captured cell from the particles. Morphological and biochemical analysis of the processed tumor cells reveal that the cells did not alter their behavior compared to the control cells, and the released cells could be successfully sub-cultured for more than two weeks. Thus, the t-MNPs do not change the cell physiology or induce any stress to the cells during the process of capture and release. The t-MNP system described herein represents a versatile approach, and can be used with different targeting ligands, such as antibodies, antibody fragments, peptides, glycoproteins, polysaccharides, and nucleic acid aptamers. The ligands may be modified by the addition of a thiol-containing group such as cysteine. Additionally, chemicals or carbohydrate-based ligands can also be used after attachment of a thiol group to a terminal end of the ligand.

### EXAMPLES

### Example 1

### Preparation and Characterization of Au@Gelatin@PCL-SPION Microparticles

Super paramagnetic iron oxide (Fe₂O₃) nanoparticles (SPIONs) were synthesized by co-precipitation of ferrous (Fe²) and ferric (Fe^{3±}) ions using a base (ammonium hydroxide). The as synthesized SPIONs were purified by magnetic separation in an N₂ environment and were surface stabilized by treating with oleic acid in chloroform to obtain hydrophobic SPIONs. The oleic acid capped SPIONs were washed twice with chloroform to remove excess oleic acid, and were stored at 4° C. until use. The hydrophobic SPIONs were encapsulated inside the hydrophobic core of poly(epsilon caprolactone) (PCL) microparticles by homogenization at 8000 rpm for 10 min using cetyltrimethylammonium bromide (CTAB) as surface stabilizer. The SPION encapsulated PCL particles thus formed were centrifuged twice at 4000 rpm for 10 min followed by washing in MilliQ® water to remove excess CTAB.

The washed particles were imaged under TEM to confirm encapsulation of SPIONs in the core (FIG. 3). TEM analysis revealed that the PCL-SPION particles were in the range of 600-800 nm, spherical in shape and had a uniform distribution of SPIONs within the hydrophobic PCL core. The response of the PCL-SPION particles to an external magnetic field was also confirmed using a small handheld magnet of the type that is routinely available for household or laboratory use, and the particles were found to be highly responsive to the magnetic field.

The PCL-SPION particles were then suspended in a 200 mg solution of gelatin in 20 ml MilliQ® water, and stirred at 3000 rpm overnight at 4° C. to facilitate the formation of a gelatin coating on the surface. The gelatin-coated PCL-SPION particles were washed twice with phosphate buffered saline (PBS, pH 7.4) to remove excess gelatin, and were purified each time by magnetic purification.

Borohydride-reduced gold nanoparticles were synthesized by adding 10 mg of sodium borohydride to an aqueous solution of 10⁻⁴ M HAuCl₄ at room temperature, followed by rigorous mixing of the solution. The pale yellow color gold ion solution instantly turned deep orange in color, confirming the reduction of ions. The solution was further incubated overnight at room temperature to allow complete reduction, and was purified by centrifugation at 13000 rpm for 1 h. The gold nanoparticles were characterized by UV-vis-NIR spectroscopy and showed a sharp absorption peak at 520 nm, characteristic of spherical nanoparticles (FIG. 3D). TEM analysis of the gold nanoparticles revealed uniformly sized spherical nanoparticles of 3-4 nm diameter (FIG. 3C).

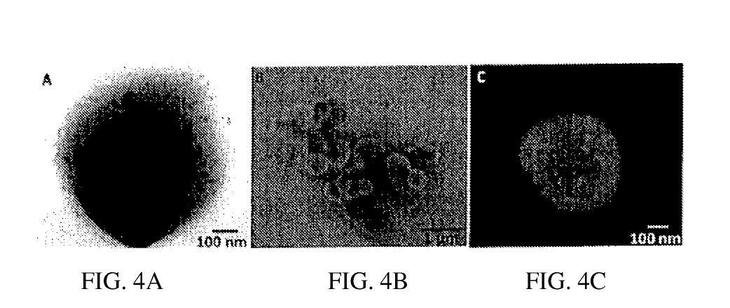

Gelatin-coated PCL-SPION particles were incubated with gold nanoparticles, prepared as described above, at room temperature for 24 h with stirring. The Au@ Gelatin@PCL-SPION particles obtained were purified by magnetic separation and observed under TEM and SEM (FIG. 3B). The TEM images revealed a uniform coating of gold nanoparticles on the surface of gelatin-coated PCL-SPION particles (FIG. 4A). The SEM images showed uniformly spherical Au@Gelatin@PCL-SPION particles but did not show the surface coated with Au nanoparticles because the size of the nanoparticles was beyond the resolution of SEM (FIG. 4B & 4C).

### Example 2

### EGFR-Specific Tumor Cell Capture System

Au @ Gelatin@ PCL-SPION magnetic nanoparticles (MNPs) were prepared as described in Example 1. To facilitate their selective binding to cancer cells through receptors overexpressed on the cell surface, epidermal growth factor receptor (EGFR) was used as an exemplary target. EGFR is overexpressed in thyroid, lung, breast, pancreatic and ovarian cancer cells. EGFR targeting peptide GE11, having the amino acid sequence YHWYGYTPQNVIGGGGC (SEQ ID NO:1), was immobilized on the gold nanoparticles bound to the gelatin-coated PCL-SPION particles using the thiol group of the terminal cysteine on the peptide. The Au-thiol linkage was selected for binding of the peptide because the thiol linkage is not as strong as many other covalent bonds and is readily broken or displaced in the presence of a high concentration of a competing thiol-containing ligand such as glutathione.

MNPs were incubated with a 2 mg/ml concentration of the GEl 1 peptide in PBS (pH-7.4) overnight with slow stirring at room temperature. The peptide-functionalized targeted MNP (t-MNP) were purified by magnetic separation followed by washing with PBS to remove loosely bound peptide. The t-MNPs were stored at 4° C. until use for capturing cancer cells of choice.

### Example 3

### Isolation of EGFR-Expressing Panc-1 Cells from Blood

Luciferase expressing Panc-1 cells (Panc-1 (luc)) were chosen as a model target for studying cell capture and release. Panc-1 cells are known to highly overexpress EGF receptors on their surface. Further, luciferase-expressing Panc-1 cells can be easily distinguished from any nonspecific ally bound contaminating cells based on the bioluminescence resulting from the activity of luciferase.

Cells were grown to 80% confluency in T75 flasks with complete DMEM (Dulbecco's modified Eagle Medium) containing 10% FBS (fetal bovine serum) and 1% penicillin-streptomycin. The cells were detached by treating with 3 mL trypsin-EDTA for 2 minutes, neutralized with 9 mL of medium, centrifuged at 1000 rpm for 10 min and resuspended in 10 mL fresh medium. The cell were counted using hemacytometer, and a known number of cells were diluted into normal whole mouse blood for analysis of cell capture and release.

### Example 4

### Cell Capture and Release

Whole blood was withdrawn from nu/nu mice by cardiac puncture into EDTA (ethylenediaminetetraacetic acid) treated sterile micro centrifuge tubes, and 500 μL blood was mixed with varying numbers of Pane-1 (luc) cells. The tubes were gently mixed using a vortex mixer to suspend the cells uniformly, and 200 μL of t-MNPs were added. The t-MNPs were allowed to bind to the cancer cells in the blood for 15 min at room temperature, and were then extracted to the tube walls by applying an external magnetic field. The blood was discarded from the centrifuge tubes, and the particles were washed twice with sterile PBS to remove any loosely/non-specifically bound cells. The washed particles were incubated in 1 mL cell culture medium containing 2 mg/ml glutathione to remove EGFR binding peptide from the MNPs, thus aiding release of the MNPs from the cell surface. The released MNPs were extracted to the wall of the centrifuge tube using external magnetic field, and the media containing the free cells were plated into 6-well plates, and supplemented with 1 mL of medium. The cells were incubated overnight at 37° C. under 5% CO₂ to facilitate attachment to the well surface, and were observed under light microscope. Microscopic assessment confirmed that the cells extracted from the spiked blood samples were morphologically similar to the control Pane-1 luc cells (FIG. 5). The efficiency of cell harvesting achieved using the polymeric micro-magnets is shown in a histogram plot in FIG. 7.

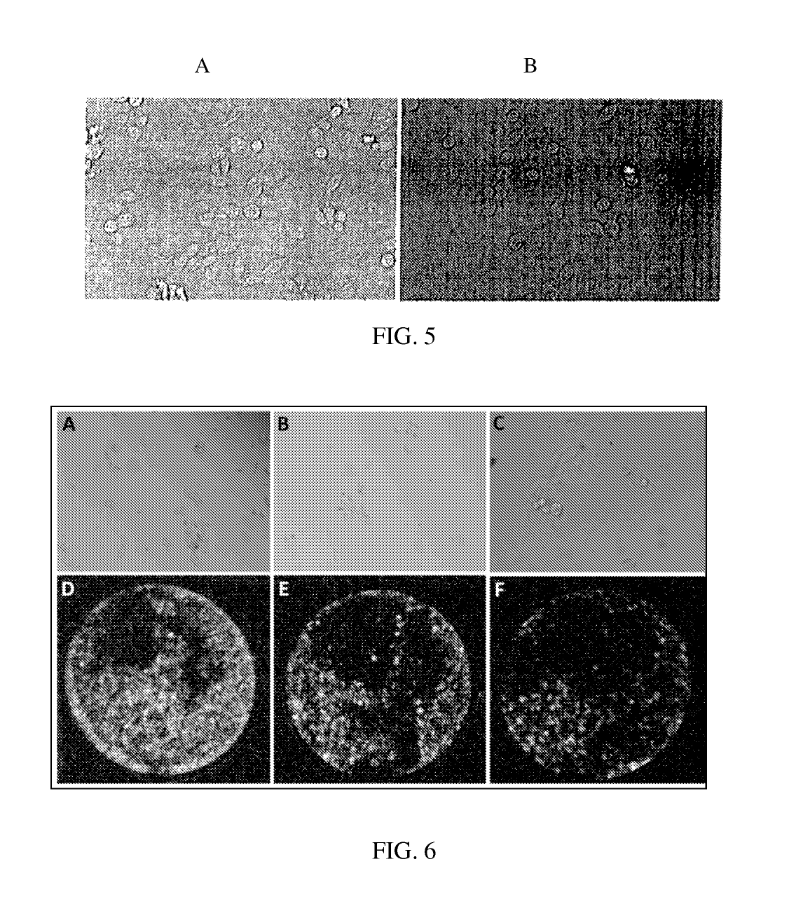

The cells captured and released from t-MNPs were further sub-cultured for 1 week with change of medium every 2 days, to ensure that they were healthy and physiologically active. Monitoring of the cells with a microscope demonstrated that the cells were indeed healthy, and divided rapidly while maintaining their original morphology and cell cycle. FIGS. 6A-6C show Panc-1 (luc) cells captured and released from 500 μL of blood spiked with 50,000, 5,000 and 500 cells, respectively, followed by sub-culturing for one week. The microscopic images clearly show that the cells maintain their morphology and have divided to form clusters on the plate surface.

The captured cells were tested for luciferase activity to ascertain their identity as Panc-1 (luc) cells. A positive identification would confirm that the t-MNPs prepared and used as described above specifically capture the target cells from a pool of blood cells and proteins. The captured and released cells plated in 6-well plates were exposed to 3 mg/ml concentration of luciferin in PBS (pH-7.4), the substrate of luciferase. Luciferase catalyzes the substrate to produce bioluminescence, and was used as a molecular marker to identify the cell of interest. In the presence of the substrate, the cells showed a very strong bioluminescence signal, confirming that the t-MNPs specifically captured Panc-1 (luc) cells (FIGS. 6D-6F).

### Example 5

### CTC Capture from Blood Isolated from Metastatic Panc-1 Tumor-Bearing SCID Beige Mice

Establishment of metastatic SCID beige mice pancreatic tumor model

Panc-1 (luc) cells are used to characterize CTC capture in a pancreatic metastatic tumor model in SCID beige mice. Cell are cultured in Dulbecco's Modified Eagle's Medium (DMEM) supplemented with 10% heat inactivated FBS and 1% Pen/Strep combined antibiotics. SCID beige mice (4-6 weeks old) are anesthetized using 2% isoflurane, injected subcutaneously with preemptive analgesic, cleaned in the surgical area with 70% alcohol, shaved, and sterilized with betadine. A small subcostal incision (<1 cm) is made and 1×10⁶ Panc-1 (luc) cells are injected at the tip of the exteriorized spleen. This procedure has been reported to lead to the development of malignant tumors in 2-3 weeks post-surgery (Little et al., 2012). Secondary tumor development indicating metastasis is affirmed by in vivo luciferin bioluminescence assay. On confirmation of metastasis, blood from the animals is drawn through cardiac puncture and assessed for capture and release of CTCs.

Analysis of Performance of Polymeric Micro-magnets as Compared to CellSearch®

The CTCs capture efficiency of polymeric micro-magnets is compared to the FDA-approved CellSearch® system. Blood samples from same mice are evaluated for CTC levels using the CellSearch® system as well as the polymeric micro-magnets described herein to assess the relative performance level. The captured CTCs are released from the polymeric micro-magnets using a competitive ligand, sub-cultured, and their viability, morphology and molecular fingerprints analyzed.

### REFERENCES

Allard, W. J., Matera, J., Miller, M. C., Repollet, M., Connelly, M. C., Rao, C., Tibbe, A. G., Uhr, J. W., and Terstappen, L. W. Tumor cells circulate in the peripheral blood of all major carcinomas but not in healthy subjects or patients with nonmalignant diseases. Clin Cancer Res. 10:6897-6904 (2004)
Braun, S. , Hepp, F., Sommer, H. L., and Pantel, K. Tumor-antigen heterogeneity of disseminated breast cancer cells: implications for immunotherapy of minimal residual disease. Int J Cancer. 84:1-5 (1999)
Chawlaand, J. S., and, Amiji, M. M. Biodegradable poly(epsilon -caprolactone) nanoparticles for tumor-targeted delivery of tamoxifen. Int J Pharm. 249:127-138 (2002)
Chawlaand, J. S., and Amiji., M. M. Cellular uptake and concentrations of tamoxifen upon administration in poly(epsilon-caprolactone) nanoparticles. AAPS Pharm Sci. 5:E3 (2003).
Cohen, S. J., Punt, C. J., Iannotti, N., Saidman, B. H., Sabbath, K. D., Gabrail, N. Y., Picus, J., Morse, M., Mitchell, E., Miller, M. C., Doyle, G. V. , Tissing, H., Terstappen, L. W., and Meropol, N. J. Relationship of circulating tumor cells to tumor response, progression-free survival, and overall survival in patients with metastatic colorectal cancer. J Clin Oncol. 26:3213-3221 (2008)
Danila, D. C., Fleisher, M., and Scher, H. I. Circulating tumor cells as biomarkers in prostate cancer. Clin Cancer Res. 17:3903-3912 (2011)
Farace, F., Massard, C., Vimond, N., Drusch, F., Jacques, N., Billiot, F., Laplanche, A., Chauchereau, A., Lacroix, L., Planchard, D., Le Moulec, S., Andre, F., Fizazi, K., Soria, J. C., and Vielh, P. A direct comparison of CellSearch and ISET for circulating tumour-cell detection in patients with metastatic carcinomas. Br J Cancer. 105:847-853 (2011)
Fizazi, K., Morat, L., Chauveinc, L., Prapotnich, D., De Crevoisier, R., Escudier, B., Cathelineau, X., Rozet, F., Vallancien, G., Sabatier, L., and Soria, J. C. High detection rate of circulating tumor cells in blood of patients with prostate cancer using telomerase activity. Ann Oncol. 18:518-521 (2007)
Husemann, Y., Geigl, J. B., Schubert, F., Musiani, P., Meyer, M., Burghart, E., Forni, G., Eils, R., Fehm, T., Riethmuller, G., and Klein, C. A. Systemic spread is an early step in breast cancer. Cancer Cell. 13:58-68 (2008)
Little, E. C., Wang, C., Watson, P. M., Watson, D. K., Cole, D. J., and Camp, E. R. Novel immunocompetent murine models representing advanced local and metastatic pancreatic cancer. J Surg Res. 176:359-366 (2012).
Litvinov, S. V., van Driel, W., van Rhijn, C. M., Bakker, H. A., van Krieken, H., Fleuren, G. J., and Warnaar. S. O. Expression of Ep-CAM in cervical squamous epithelia correlates with an increased proliferation and the disappearance of markers for terminal differentiation. Am J Pathol. 148:865-875 (1996)
Livand, G.-y., and Salmeron, M. B. Reversible displacement of chemisorbed n-alkanethiol molecules on Au (111) surface: an atomic force microscopy study. Langmuir. 10:367-370 (1994)
Muller, V., Stahmann, N., Riethdorf, S., Rau, T., Zabel, T., Goetz, A., Janicke, F., and Pantel, K. Circulating tumor cells in breast cancer: correlation to bone marrow micrometastases, heterogeneous response to systemic therapy and low proliferative activity. Clin Cancer Res. 11:3678-3685 (2005)
Nagrath, S., Sequist, L. V., Maheswaran, S., Bell, D. W., Irimia, D., Ulkus, L., Smith, M. R., Kwak, E. L., Digumarthy, S., Muzikansky, A., Ryan, P., Balis, U. J., Tompkins, R. G., Haber, D. A., and Toner, M. Isolation of rare circulating tumour cells in cancer patients by microchip technology. Nature. 450:1235-1239 (2007)
Nakagawa, T. , Martinez, S. R., Goto, Y., Koyanagi, K., Kitago, M., Shingai, T., Elashoff, D. A., Ye, X., Singer, F. R., Giuliano, A. E., and Hoon, D. S. Detection of circulating tumor cells in early-stage breast cancer metastasis to axillary lymph nodes. Clin Cancer Res. 13:4105-4110 (2007)
Paterlini-Brechotand, P., Benali. N. L. Circulating tumor cells (CTC) detection: clinical impact and future directions. Cancer Lett. 253:180-204 (2007)
Rezwan, K., Chen, Q. Z., Blaker, J. J., and Boccaccini, A. R. Biodegradable and bioactive porous polymer/inorganic composite scaffolds for bone tissue engineering. Biomaterials. 27:3413-3431 (2006)
Shenoyand, D. B., and Amiji, M. M. Poly(ethylene oxide)-modified poly(epsilon-caprolactone) nanoparticles for targeted delivery of tamoxifen in breast cancer. Int J Pharm. 293:261-270 (2005)
Sidransky. D., Emerging molecular markers of cancer. Nat Rev Cancer. 2:210-219 (2002)
Stott, S. L., C. H. Hsu, D. I. Tsukrov, M. Yu, D. T. Miyamoto, B. A. Waltman, S. M. Rothenberg, A. M. Shah, M. E. Smas, G. K. Korir, F. P. Floyd, Jr., A. J. Gilman, J. B. Lord, D. Winokur, S. Springer, D. Irimia, S. Nagrath, L. V. Sequist, R. J. Lee, K. J. Isselbacher, S. Maheswaran, D. A. Haber, and M. Toner. Isolation of circulating tumor cells using a microvortex-generating herringbone-chip. Proc Nail Acad Sci U S A. 107:18392-18397 (2010)
Thurm, H., Ebel, S., Kentenich, C., Hemsen, A., Riethdorf, S., Coith, C., Wallwiener, D., Braun, S., Oberhoff, C., Janicke, F., and Pantel, K. Rare expression of epithelial cell adhesion molecule on residual micrometastatic breast cancer cells after adjuvant chemotherapy. Clin Cancer Res. 9:2598-2604 (2003)

1. A releasable targeted magnetic microparticle for use in capturing cells of a targeted type, the microparticle comprising:
a plurality of hydrophobic superparamagnetic nanoparticles embedded in a hydrophobic polymer to form a hydrophobic core microparticle;
a coating comprising a biopolymer surrounding the core microparticle;
a plurality of targeting nanoparticles attached to an outer surface of the coating, wherein the targeting nanoparticles comprise a releasable targeting moiety on their surface.

2. The magnetic microparticle of claim 1, wherein the superparamagnetic nanoparticles comprise a material selected from iron, cobalt, nickel, salts or oxides thereof, and combinations thereof.

3. The magnetic microparticle of claim 1, wherein the hydrophobic polymer is PCL.

4. The magnetic microparticle of claim 1, wherein the biopolymer is thiolated gelatin.

5. The magnetic microparticle of claim 1, wherein the releasable targeting moiety is selected from the group consisting of: amino acids, peptides, proteins, sugars, oligosaccharides, nucleic acids, nucleotides, oligonucleotides, and aptamers.

6. The magnetic microparticle of claim 5, wherein the releasable targeting moiety is EGF or a derivative of EGF that binds to an EGF receptor.

7. The magnetic microparticle of claim 1, wherein the targeting moiety is coupled to the targeting nanoparticles by a thiol linkage.

8. The magnetic microparticle of claim 7, wherein the microparticle is releasable from its target cell by a disulfide bond disruptive agent.

9. The magnetic microparticle of claim 8, wherein the disulfide bond disruptive agent is glutathione.

10. The magnetic microparticle of claim 1, wherein the microparticle is targeted to a circulating tumor cell.

11. The magnetic microparticle of claim 1, wherein the targeting nanoparticles comprise gold.

12. A composition comprising a plurality of the magnetic microparticles of claim 1.

13. The composition of claim 12, further comprising a physiological buffer solution in which the magnetic microparticles are suspended.

14. A composition comprising the releasable magnetic microparticle of claim 1 bound to a targeted cell.

15. A method of isolating a cell, the method comprising the steps of:
providing the composition of claim 12,
contacting the composition with a fluid comprising individual cells in suspension, whereby targeted cells bind to said magnetic microparticles; and
isolating the magnetic microparticles and bound cells using a magnet.

16. The method of claim 15, wherein the targeted cells are circulating tumor cells and the fluid is a bodily fluid from a mammalian subject.

17. The method of claim 16 wherein the subject is a human cancer patient.

18. The method of claim 15, wherein the superparamagnetic nanoparticles comprise iron oxide, the hydrophobic polymer comprises PCL, the biopolymer coating comprises thiolated gelatin, and the microparticle is releasable from its target cell by a disulfide bond disruptive agent.

19. A method of fabricating the composition of claim 12, the method comprising the steps of:
(a) providing a plurality of hydrophobic superparamagnetic nanoparticles;
(b) embedding the nanoparticles of (a) in a matrix of hydrophobic polymer to form microparticles comprising the nanoparticles;
(c) coating the microparticles from (b) with a biopolymer to form a coating of the biopolymer surrounding the microparticles;
(d) attaching a plurality of targeting nanoparticles to the biopolymer coating of (c) to form the composition of claim 12, wherein the targeting nanoparticles comprise a plurality of targeting moieties attached to their surface.

20. The method of claim 19, further comprising washing and/or isolating the microparticles of any of steps (b)-(d) using a magnet.

21. The method of claim 19, further comprising suspending the microparticles resulting from step (d) in a physiological buffer solution.

22. A microparticle comprising a plurality of hydrophobic superparamagnetic nanoparticles embedded in a hydrophobic polymer.

23. The microparticle of claim 22, wherein the nanoparticles are uniformly distributed within the microparticle.

24. The microparticle of claim 22, wherein the nanoparticles comprise a material selected from iron, cobalt, nickel, salts or oxides thereof, and combinations thereof.

25. The microparticle of claim 22, wherein the nanoparticles comprise iron oxide.

26. The microparticle of claim 22, wherein the hydrophobic polymer is PCL.

27. A composition comprising a plurality of the microparticle of claim 22.

28. A kit comprising the composition of claim 12 and one or more reagents and/or instructions for use.

29. (canceled)

30. A plurality of the microparticles of claim 1 having an average particle diameter in the range from about 500 nm to about 1000 nm.

31. The microparticle of claim 30 having an average particle diameter in the range from about 600 nm to about 800 nm.
